SEQUENCE LISTING (4 sequences)

SEQ ID NO: 1 - DNA, 31 bp, Artificial Sequence
  Description of Artificial Sequence Synthetic probe
agaacacgccactattcacacgcgcgtatgc

SEQ ID NO: 2 - DNA, 36 bp, Artificial Sequence
  Description of Artificial Sequence Synthetic probe
atcgcccgcacgctcacagttaagccgtgagatttc

SEQ ID NO: 3 - DNA, 25 bp, Artificial Sequence
  Description of Artificial Sequence Synthetic probe
acacacgctcgcgcgcgatacgccc

SEQ ID NO: 4 - DNA, 25 bp, Artificial Sequence
  Description of Artificial Sequence Synthetic probe
actacaccccaaaggggatgcgccc

(19) United States
(12) Patent Application Publication
(10) Pub. No.: US 2012/0264119 A1
(43) Pub. Date: Oct. 18, 2012

(54) NEW METHOD FOR DECONTAMINATION AND PROCESSING OF CLINICAL SPECIMENS FROM A PATIENT

(76) Inventors: Jyotsna S. Shah, Santa Clara, CA (US); Helena Weltman, Los Altos, CA (US); Patricia Narciso, Union City, CA (US)

(21) Appl. No.: 13/371,711

(22) Filed: Feb. 13, 2012

Related U.S. Application Data
(60) Provisional application No. 61/463,128, filed on Feb. 14, 2011.

(51) Int. Cl. C12Q 1/04; G01N 21/64; C07H 21/00; C12Q 1/68

(52) U.S. Cl. 435/6.11; 435/34; 435/6.12; 536/23.1

(57) ABSTRACT

The present invention relates to a new method for decontaminating and processing clinical samples suspected of containing Mycobacteria. In this method the clinical samples are processed in a solution comprising a hypochlorite salt and N-acetyl cysteine.

This application cites priority to U.S. Provisional Application No. 61/463,128, filed Feb. 14, 2011.

### BACKGROUND

The century old sputum microscopy test using Ziehl-Neelsen stained smears form unconcentrated sputum (direct smears) remains the primary tool for diagnosing tuberculosis (TB) in low-income countries. However direct smear microscopy has poor sensitivity (range 20-80%) particularly in HIV-coinfected patients (Steingart, K. R., V. Ng, M. Henry, P. C. Hopewell, A. Ramsay, J. Cunningham, R. Urbanczik, M. D. Perkins, M. A. Aziz, and M. Pai. 2006. Sputum processing methods to improve the sensitivity of smear microscopy for tuberculosis: a systematic review. Lancet Infect. Dis. 6:664-674). Therefore, the Stop TB Partnership Retooling Task Force identified bleach sedimentation as one of the 3 promising approaches to improving the sensitivity of sputum smear microscopy in high burden countries (World Health Organization. 2008. New laboratory diagnostic tools for tuberculosis control. Stop TB Partnership Retooling Task Force. WHO Press, Geneva, Switzerland). The biggest problem with this technique is that mycobacteria become non-viable and, therefore, cannot be cultured from the bleach treated sputum.

Further, recent reviews regarding use of bleach question whether bleach processing really increases the accuracy of sputum smear microscopy for diagnosis of pulmonary tuberculosis. (A. Cattamanchi, J. L. Davis, M. Pai, L. Huang, P. C. Hopewell and K. R. Steingart. 2010. Does Bleach processing increase the accuracy of sputum smear microscopy for diagnosing pulmonary tuberculosis? J. Clin. Microbiol. 48:2433-2439).

In middle and high income countries processing of sputum by chemical and/or physical methods is commonly used to increase diagnostic sensitivity. The most commonly used methods use sodium hydroxide alone or a combination of sodium hydroxide and N-acetyl-cysteine. The problem with this method, although the best at this point in time for concentration of sputum, destroys more than 80% of the viable mycobacteria due to use of NaOH.

### SUMMARY OF THE INVENTION

The present invention provides a method for decontamination, digestion and concentration of clinical specimens (samples) from a patient including, but not limited to, sputum, bronchial lavage sample (Bronchoalveolar lavage: BAL), induced sputum, gastric washings, urine, fecal material in suspension, ground tissue (including ground skin), peritoneal fluid and pleural fluid for detection of mycobacteria using a combination of a mucolytic reagent such as an acetyl-cysteine or chitin and sodium or calcium hypochlorite (commonly known as bleach). This combination of reagents protects nucleic acids (DNA and RNA) and at the same time selectively kills non-mycobacterial pathogens that interfere with growth of mycobacterial cultures. The processed concentrated pellet can be tested for mycobacteria directly by Fluorescent in Situ Hybridization (FISH), PCR, RT-PCR or by culture confirmation methods. The method is comprised of several steps which are performed in the listed order in the Exemplification Section, below.

The two most commonly used methods for decontamination and concentration of clinical samples including sputum, BAL, gastric washings, fecal material, urine, include N-acetyl cysteine and sodium hydroxide (NaOH) or sodium hydroxide only, prior to culturing of Mycobacteria. The problem with these methods is that sodium hydroxide destroys RNA. Therefore, the viability of the cells varies and the ability to detect target sequences varies depending on, for example, how well the pellet is washed and the final pH of the pellet. It can be seen then how this procedure often can result in substandard results. When samples were tested by Mycobacteria Genus specific Fluorescent in Situ hybridization (FISH) assays, it was observed that the sensitivity of the NaOH/Nalc processed pellet was always lower than the acid-fast (Ziehl-Neelsen) staining.

Another method that has been used to detect mycobacteira in sputum includes processing sputum with sodium hypochlorite (bleach) solution (between 2% to 10%). Unfortunately, the cells are not viable after bleach treatment.

Therefore we have developed a new method using a combination of N-acetyl-cysteine and bleach. In this method an equal volume (or approximately 1-3 volumes) of a solution containing a hypochlorite salt (approximately 0.5% to approximately 2%) including but not limited to sodium hypochlorite (bleach) or calcium hypochlorite and (approximately 0.5 to approximately 2%) N-acetyl cysteine is added to sputum, BAL or other suitable sample, mixed by vortexing and incubated at room temperature for 15-40 minutes, preferably 30 minutes. In a preferred embodiment of the present invention, processing solution contains 1% sodium hypochlorite and 1% N-acetyl cysteine. Ratio of sputum to processing solution is approximately 1:1 by volume.

More than one type of hypochlorite salt (i.e., that is, a mixture of two or more) may be used at the same time providing the total concentration does not exceed the concentration range given above. After incubation the solution is then centrifuged at approximately 3000 g for approximately 15 minutes. One of skill in the art will understand that centrifuge speed and centrifuge time are inversely related and that faster centrifuge speeds will allow for shorter centrifuge times. The supernatant is decanted and the pellet is, optionally, washed once with water. The washed pellet is resuspended in approximately 1/10 the volume of water (or physiological saline solution) of the starting sample volume and mixed by vortexing. The resuspended pellet is then tested directly by PCR RT-PCR, FISH or other suitable assay known to those of skill in the art or the mycobacteria can be cultured.

### EXEMPLIFICATION

We compared sensitivity of the prior art Mycobacteria FISH assay with acid fast (Ziehl-Neelsen) staining of processed pellets. See, Table 1. Although the prior art FISH assay detected all the acid-fast positive smears, the grading sensitivity was statistically lower for FISH assay as compared to the acid-fast smear sample by almost one log, clearly demonstrating that more than 80% of the cells are destroyed during processing. This led us to develop a new method that not only concentrates the mycobacteria in sputum but also does not destroy RNA, as demonstrated below.

A non-limiting exemplification of the present method comprises:
1. To the clinical sample in a tube (direct or resuspended in buffer or broth), add an equal volume (or 1-3 volumes) of a processing solution containing N-acetyl cysteine or chitin and a salt containing hypochlorite. The processing solution is mixed as follows: A solution containing a hypochlorite salt (approximately 0.5% to approximately 2%) including but not limited to sodium hypochlorite (bleach) or calcium hypochlorite and (approximately 0.5 to approximately 2%) N-acetyl cysteine. Mix the clinical sample/processing solution well by vortexing or other suitable method. The solution (composition) of the present invention detailed above may, without limitation, comprise, consist essentially of or consist of a salt containing hypochlorite (hypochlorite salt) and N-acetyl cysteine. One of ordinary skill in the art will be able to suitably modify the composition of the present invention in view of the teachings of the present specification.
2. Incubate at room temperature for about 20-30 (about 15-40) minutes and, optionally, mix occasionally by inverting the tube several times.
3. Centrifuge the tube at approximately 5000 g for about 10 minutes (or approximately 3000 g, which is standard in most labs, for about 15 minutes). Optionally, wash the pellet with water and centrifuge at 3000 g for 15 minutes.
4. Resuspend the pellet in approximately 0.5 to 1.00 ml of water.
5. The pellet can be tested for the presence of Mycobacteria by acid-fast staining, FISH, PCR, RT-PCR.
6. Additionally or alternatively, culture Mycobacteria either on solid or liquid media, followed by culture confirmation using biochemical or nucleic acid-based assays.
7. Physiological saline may be substituted for water.

For the data presented below, sputum was processed with an equal volume of the processing solution containing N-acetyl cysteine and sodium or calcium hypochlorite. The processing solution is mixed as follows: sodium hypochlorite (bleach; approximately 1.0%) and N-acetyl cysteine (approximately 1.0%). Briefly, the sputum was mixed with the processing solution, vortexed and incubated at room temperature for 20 to 30 minutes with, optionally, occasional mixing by inverting the tubes several times. After incubation the tubes were centrifuged at about 5000 g for about 10 minutes. The supernatant was removed. The pellet was washed once with water. The washed pellet was resuspended in 0.5 ml of water and an aliquot was tested by FISH assays (M-Genus, Tb-FISH, MAC-FISH, Kan-FISH), acid-fast staining and culture. FISH assays and appropriate probes for the detection of Mycobacterium are known to those of ordinary skill in the art. The use of any suitable probes or probes is contemplated by the present invention. The acid-fast stain prepared from processed sputum was compared to acid-fast smear prepared with processed pellet using either N-acetyl-cysteine/NaOH. Results are summarized below in Tables 1 and 2.

Sputum or induced sputum samples from patients suspected of tuberculosis (Table 1) and spiked sputum or induced sputum samples with M. avium, M. kansasii and M. gordonae (Table 2) were processed as described above. Sputum or induced sputum was processed with equal volumes of a processing solution containing N-acetyl cysteine and sodium or calcium hypochlorite (processing solution). Briefly, the sputum was mixed with the processing solution, vortexed and incubated at room temperature between 20 to 30 min, with occasional mixing by inverting the tubes several times. After incubation the tubes were centrifuged at 3000 g for 15 minutes. Supernatant was removed. The pellet was washed once with water. The washed pellet was resuspended in 0.5 ml of water and an aliquot was tested by FISH assays for detection an speciation of mycobacteria to species level (M. tuberculosis complex (TB), M. avium complex (MAC), M. kansasii (M. kan), acid-fast staining and culture. The acid-fast stain prepared from sputum processed as above was compared to acid-fast smear prepared with processed pellet using either N-acetyl-cysteine/NaOH or NaOH. Results are summarized below in Table 1 and Table 2. In addition, an aliquot of the processed pellets after neutralization were cultured on LJ slants (Lowenstein-Jensen medium slants) and confirmed by FISH assays.

FISH Assays: Methanol fixed smears prepared from processed sputum or culture were tested by FISH assays. The methanol fixed smears were hybridized with mycobacteria genus (M-genus) probes for 30 min at 37 ° C. Excess probe was removed by washing the smear 3 times with a low salt buffer. The smears were completely dried and viewed under fluorescent microscope at 1000× magnification using specific filters. If the sample was positive for M-genus, the bacteria present in the smear gave a green signal. All the samples that were positive by the M-genus test were further tested by TB-FISH, using MTB complex specific probe, MAC-FISH using MAC specific probe, M. kansasii FISH, using M. kansasii specific probe. Results are presented in Table 1 and Table 2. Table 1 shows results from clinical samples. Table 2 shows results from negative sputum samples spiked with mycobacteria. Spiked samples were used in Table 2 to provide controlled conditions and to ensure consistency between conditions.

TABLE 1

| | Method | | | | | | | |
|---|---|---|---|---|---|---|---|---|
| | NaOH/- | | | | Sodium or calcium hypochlorite - | | | |
| | N-acetyl cysteine | | | | N-acetyl cysteine | | | |
| | | M-genus | | | | M-genus | | |
| Sample # | AFB | FISH | TB-FISH | Culture | AFB | FISH | TB-FISH | Culture |
| 1 | 4+ | 2+ | 2+ | TB | 4+ | 4+ | 4+ | TB |
| 2 | 4+ | 3+ | 3+ | TB | 4+ | 4+ | 4+ | TB |
| 3 | 1+ | Neg | Neg | TB | 2+ | 2+ | 2+ | TB |

TABLE 2

| | METHOD: | | | | | | | | | | | |
|---|---|---|---|---|---|---|---|---|---|---|---|---|
| | NaOH/Nalc processed sputum, spiked | | | | | | NaClO/Nalc processed sputum, spiked | | | | | |
| | with cultured mycobacteria | | | | | | with cultured mycobacteria | | | | | |
| | | Mycobacterium FISH assays | | | | Culture of | | Mycobacterium FISH assays | | | | Culture of |
| | | | | M- | | processed | | | | M- | | processed |
| SPIKED | | M- | | avium | M- | cell | | M- | | avium | M- | cell |
| NEGATIVE | | Genus- | TB- | complex - | kansasii - | pellet on | AF | Genus- | TB- | complex- | kansasii - | pellet on |
| SPUTUM | AFB | probe | probe | probe | probe | LJ slants | B | probe | probe | probe | probe | LJ slants |
| M. KANSASII- | 4+ | 1+ | 0 | 0 | 1+ | M | 4+ | 4+ | 0 | 0 | 4+ | M |
| SPIKED | | | | | | kansasii | | | | | | kansasii |
| | | | | | | growth | | | | | | growth |
| | | | | | | after 3 | | | | | | after 3 |
| | | | | | | weeks, | | | | | | weeks, |
| | | | | | | confirmed | | | | | | confirmed |
| | | | | | | by FISH | | | | | | by FISH |
| M AVIUM | 4+ | 0 | 0 | 0 | 0 | Mac | 4+ | 4+ | 0 | 4+ | 0 | Mac |
| COMPLEX- | | | | | | growth | | | | | | growth |
| SPIKED | | | | | | after 2 | | | | | | after 2 |
| | | | | | | weeks, | | | | | | weeks, |
| | | | | | | confirmed | | | | | | confirmed |
| | | | | | | by FISH | | | | | | by FISH |
| M GORDONAE - | 4+ | 1+ | 0 | 0 | 0 | N/A | 4+ | 4+ | 0 | 0 | 0 | N/A |
| SPIKED | | | | | | | | | | | | |
| NOT SPIKED | 0 | 0 | 0 | 0 | 0 | N/A | 0 | 0 | 0 | 0 | 0 | N/A |

As shown in both the tables the sensitivity of the NAlCO/NalC was much better than Nalc/NaOH processing method. Clearly the rRNA was better preserved with the method of the present invention over the prior art method. Secondly, mycobacteria could be cultured after processing by the method of the present invention. Additionally, these data show that the compositions and methods of the present invention work with a wide range of mycobacteria commonly found in clinical settings. AFB=acid-fast bacillus protocol of the Ziehl-Neelsen method, as is known by those of ordinary skill in the art.

Probe sequences used herein are as follows:

TB Probe
[SEQ ID NO: 1]
AGA ACA CGC CAC TAT TCA CAC GCG CGT ATG C
Mycobacterium Genus Probe
[SEQ ID NO: 2]
ATC GCC CGC ACG CTC ACA GTT AAG CCG TGA GAT TTC
M. kansasii probe
[SEQ ID NO: 3]
ACA CAC GCT CGC GCG CGA TAC GCC C
M. avium complex (MAC) probe
[SEQ ID NO: 4]
ACT ACA CCC CAA AGG GGA TGC GCC C

What is claimed is:

1. A composition suitable for decontamination and concentrating of a clinical sample for detection of Mycobacteria: the solution comprising a hypochlorite salt at a concentration of approximately 0.5% to approximately 2% and N-acetyl cysteine at a concentration of approximately 0.5 to approximately 2%.

2. composition of claim 1, wherein said hypochlorite salt is selected from a group consisting of sodium hypochlorite and calcium hypochlorite.

3. composition of claim 1, wherein said hypochlorite salt is at a concentration of approximately 1%.

4. composition of claim 1, wherein said N-acetyl cysteine is at a concentration of approximately 1%.

5. A method for decontaminating and concentrating a clinical sample for the detection of Mycobacteria, comprising:
a. adding to a clinical sample 1 to 3 volumes of a solution containing N-acetyl cysteine and a hypochlorite salt and mixing to create a mixed sample;
b. incubating the mixed sample at room temperature for approximately 15 to 40 minutes to create an incubated sample;
c. centrifuging the incubated sample to pellet the solids;
d. resuspending the pelleted solids;
e. detecting the presence of any Mycobacteria in the resuspended pellet by acid-fast staining, FISH, PCR, RT-PCR or culture.

6. method of claim 5, wherein said clinical sample is selected from a group consisting of sputum, bronchoalveolar lavage, induced sputum, peritoneal fluid, pleural fluid, ground tissue, ground skin, gastric washings, urine and fecal material in suspension.

7. method of claim 5, wherein said sample pellet of step d) is additionally washed prior to step e).

8. method of claim 5, wherein in step d) the pellet is resuspended in water.

9. method of claim 5, wherein said hypochlorite salt is at a concentration of approximately 0.5% to approximately 2% and said N-acetyl cysteine at a concentration of approximately 0.5 to approximately 2%.

10. The method of claim 5, wherein said hypochlorite salt is selected from a group consisting of sodium hypochlorite and calcium hypochlorite.

11. The method of claim 5, wherein said hypochlorite salt is at a concentration of approximately 1%.

12. The method of claim 5, wherein said N-acetyl cysteine is at a concentration of approximately 1%.

13. A composition comprising a nucleotide sequence consisting of SEQ ID NO: 3.

14. A composition comprising a nucleotide sequence consisting of a sequence complementary to SEQ ID NO: 3.

15. A composition comprising a nucleotide sequence consisting of SEQ ID NO: 4.

16. A composition comprising a nucleotide sequence consisting of a sequence complementary to SEQ ID NO: 4.
